(19) United States
(12) Patent Application Publication
(10) Pub. No.: US 2022/0015702 A1
(43) Pub. Date: Jan. 20, 2022

(54) BRAINWAVE ANALYSIS DEVICE COMPRISING A FABRIC SUPPORT ELEMENT

(71) Applicant: DREEM, Paris (FR)

(72) Inventors: Pierre EMERICH, PARIS (FR); Gabriel OPPETIT, PARIS (FR); Quentin SOULET DE BRUGIERE, PYLA SUR MER (FR)

(73) Assignee: DREEM, Paris (FR)

(21) Appl. No.: 17/414,673

(22) PCT Filed: Dec. 16, 2019
(86) PCT No.: PCT/FR2019/053094

§ 371 Date: Jun. 16, 2021

(30) Foreign Application Priority Data
Dec. 20, 2018 (FR) 18 73675

(51) Int. Cl. A61B 5/00; A61B 5/369; A61B 5/291

(52) U.S. Cl. CPC A61B 5/6804; A61B 5/369; A61B 5/6803; A61B 2562/221; A61B 2562/18; A61B 5/291

(57) ABSTRACT

A device for analysing and/or monitoring brain waves configured to be worn by a person, in particular during a period when the person is sleeping. The device includes a support element configured to at least partially surround the person's head so as to be held thereon, and the support element includes at least one three-dimensional fabric.

### TECHNICAL FIELD

The invention relates, generally, to the field of the analysis and monitoring of brain activity, and in particular the stimulation of brainwaves to reinforce the beneficial effects of a person's sleep.

### PRIOR ART

Devices are known for monitoring and stimulating the brain activity of a person, notably while he or she is sleeping, by acquiring a biological signal from the person and by stimulating brainwaves.

Such devices can take the form of a headband that can be worn on the head of the person. In particular, the headband comprises rigid and relatively bulky elements, such as cables and electronic elements, covered by a fabric covering.

### Technical Issue

However, although they do provide satisfaction, such devices can prove uncomfortable to wear, in particular while the person is sleeping.

Indeed, the rigid elements of the headband can prove a nuisance for the person during the night.

The aim of the present invention is notably to improve this situation.

In particular, the invention targets a device for analyzing and monitoring brainwaves which has little bulk and is comfortable, so as to be able to be used in an everyday life context, and which is robust and inexpensive to manufacture to ensure its accessibility to the general public.

### SUMMARY OF THE INVENTION

A device for analyzing and/or monitoring brainwaves is proposed that is configured to be worn by a person, notably during a period when the person is sleeping, the device comprising a support element configured to at least partially surround a head of the person so as to be held thereon, characterized in that the support element comprises at least one three-dimensional fabric.

By virtue of its provisions, a support element is obtained that is particularly comfortable to use.

The features set out in the following paragraphs can, optionally, be implemented. They can be implemented independently of one another or in combination with one another.

According to one embodiment, the support element comprises a plurality of three-dimensional fabric parts.

According to another embodiment, the support element is exclusively made of three-dimensional fabric.

According to another embodiment, the support element is configured to be assembled reversibly with an embedded electronics conditioning and monitoring unit.

According to another embodiment, the support element comprises at least one electrode configured to be in contact with the person to acquire at least one measurement signal representative of a physiological electrical signal of the person, the unit being configured to receive information from the electrode.

According to another embodiment, the support element comprises at least one acoustic transducer configured to emit an acoustic signal stimulating an inner ear of the person, the acoustic transducer being disposed in a three-dimensional fabric part adapted to cover the ear of the person once the support element is worn by the person, the unit being configured to control and monitor the emission by the acoustic transducer of the acoustic signal.

According to another embodiment, the unit is linked to the electrode by means of a shielded cable.

According to another embodiment, the shielded cable forms an integral part of the three-dimensional fabric.

According to another embodiment, the three-dimensional fabric forms at least one channel, the channel being configured to receive the shielded cable.

Also proposed is a kit for forming a device for analyzing and/or monitoring brainwaves as claimed in any one of the preceding claims, comprising a support element and an embedded electronics conditioning and monitoring unit, the unit being configured to be assembled reversibly with the support element.

### BRIEF DESCRIPTION OF THE DRAWINGS

Other features, details and advantages of the invention will become apparent on reading the following detailed description, and on analyzing the attached drawings, in which:

FIG. 1 shows a perspective view of a device according to an embodiment of the invention;

FIG. 2 shows a partial enlarged view of the device of [FIG. 1];

FIG. 3 shows another partial enlarged view of the device of [FIG. 1];

FIG. 4 shows a disassembled view of the device of [FIG. 1] comprising a support element and an embedded electronics conditioning and monitoring unit;

FIG. 5 shows a schematic side view of a person wearing the device of [FIG. 1].

FIG. 6 shows a schematic partial view of a three-dimensional fabric part of the device of [FIG. 1] comprising a plurality of fibers, an electrical conduction element forming an integral part of the three-dimensional fabric part; and

FIG. 7 shows an exploded view of an electrical conduction element in the form of a shielded cable, notably for linking the embedded electronics conditioning and monitoring unit to an electrode.

### DESCRIPTION OF THE EMBODIMENTS

The drawings and the description below contain, for the most part, elements of a certain nature. They can also serve not only to obtain a better understanding of the present invention, but can also contribute to the definition, thereof, as necessary.

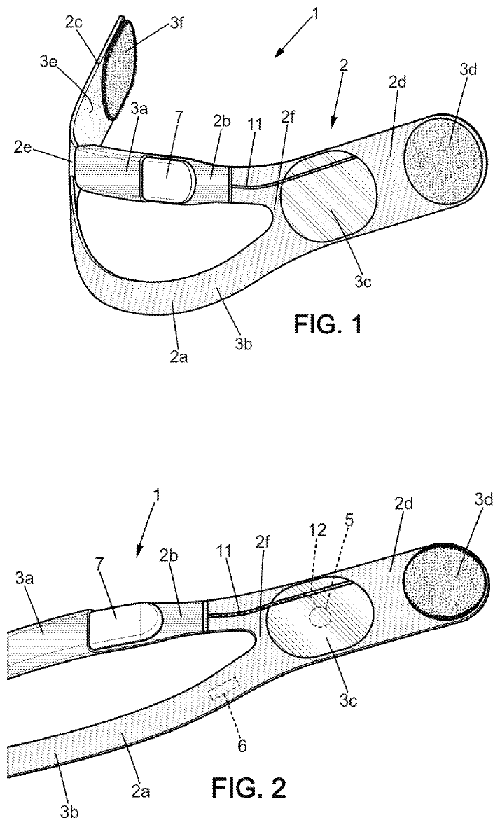

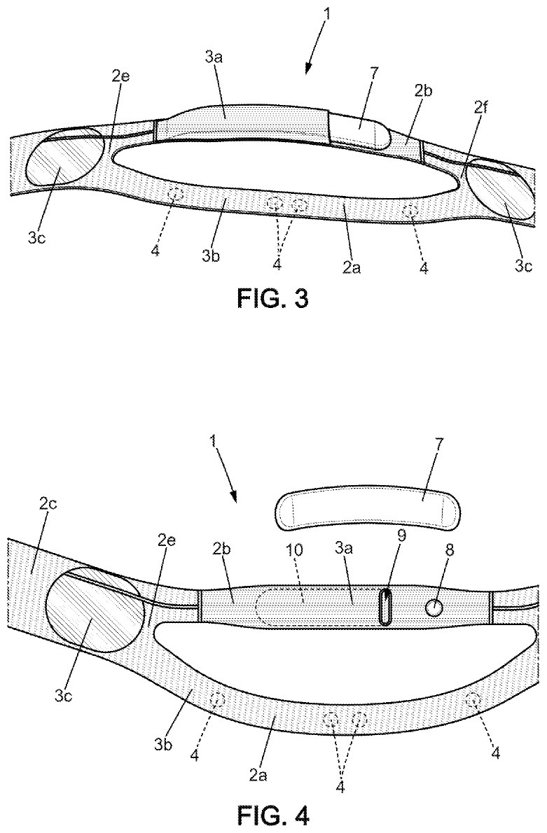

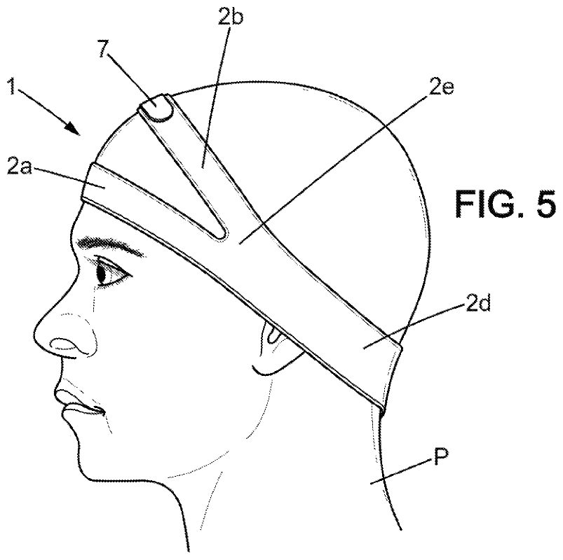

Referring to [FIG. 1], [FIG. 2], [FIG. 3], and [FIG. 4], the subject of the invention is a device 1 for analyzing and monitoring brainwaves, and in particular for stimulating brainwaves to reinforce the beneficial effects of sleep.

The device 1 is configured to be worn by a person P, notably during a period when the person is sleeping.

The device 1 is in particular configured to be worn on the head of the person P.

To this end, the device 1 comprises a support element 2, also called headband. The support element 2 is configured to at least partially surround the head of the person P so as to be held thereon.

In an embodiment of the invention illustrated in [FIG. 5], the support element 2 is notably configured to surround at least a portion of a circumference of the head of the person P, even to entirely surround the circumference of the head of the person P.

In the embodiment illustrated in [FIG. 1], the support element 2 comprises several branches 2a, 2b, 2c, 2d. The support element 2 notably comprises four branches linked to one another at branch link points 2e, 2f. The branches 2a, 2b, 2c, 2d surround different portions of the head of the person P so as to ensure a stable hold and a precise locating of the device 1 on the person P.

Thus, the first branch 2a and the second branch 2b respectively surround a front side and a top side of the head of the person P. The first and second branches 2a, 2b are respectively linked at their respective ends at a left side branch link point 2e and a right side branch link point 2f, situated in proximity to the left and right temples of the person P.

The third and fourth branches 2c, 2d respectively extend from the left side 2e and right side 2f branch link points toward the nape of the neck of the person P.

According to the invention, the support element 2 comprises one or more three-dimensional fabrics.

A “three-dimensional fabric” should be understood to mean a fabric composed of discontinuous or continuous fibers, disposed in all three directions of space. Thus a three-dimensional fabric is distinguished from a conventional fabric in that it does not extend only on a plane X-Y, but also at right angles, in a direction Z.

A fiber according to the present invention can be a natural or synthetic fiber.

A three-dimensional fabric can be produced by weaving, knitting, braiding, or be non-woven.

As a purely illustrative example, such three-dimensional fabrics are for example described in the document US 20070144221. A knitted three-dimensional fabric can notably be called “spacer fabric” or “3D knitted fabric”.

It is thus possible to produce a support element 2 that has a complex form particularly well suited to the use of the device 1 on the head of the person P. Furthermore, a three-dimensional fabric advantageously has no stitching, which allows a support element 2 to be obtained that is particularly comfortable when it is worn.

According to the embodiment of [FIG. 1], [FIG. 2], [FIG. 3], and [FIG. 4], the support element 2 comprises a plurality of three-dimensional fabric parts 3a, 3b, 3c, 3d, 3e, 3f. Each of the three-dimensional fabric parts forms a more or less extensive part of the support element 2.

Each of the three-dimensional fabric parts has specific mechanical and/or structural characteristics.

Thus, certain three-dimensional fabric parts can be relatively more rigid, or relatively more flexible, than certain other three-dimensional fabric parts.

As a variant or in combination, certain three-dimensional fabric parts 3b, 3b can have a high elasticity. These three-dimensional fabric parts 3b, 3c can thus be characterized by an elastic elongation or deformation that can be greater than 60%, even greater than 80, even greater than 90%.

It is thus possible to locally vary the mechanical behavior of the support element 2, with one of the branches 2b of the support element 2 more rigid or inextensible and the other branches 2a, 2c, 2d relatively more elastic or flexible for example.

The support element 2 also comprises a three-dimensional fabric part 3e forming an inner face of the support element 2. This three-dimensional fabric part 3e is intended to be in contact with the skin of the person P and can, to this end, be produced in a particularly comfortable and/or micro-ventilated fabric.

The support element 2 also comprises three-dimensional fabric parts 3d, 3f situated at the ends of the third and fourth branches 2c, 2d. These three-dimensional fabric parts 3d, 3f are intended to allow a fixing to one another, notably behind the nape of the neck of the person P. To this end, they can comprise a mechanical fastening system, for example by hooks or fabric loops known by the tradename “Velcro”.

The support element 2 also comprises at least one, even a plurality of, electrodes 4, illustrated by dotted lines in [FIG. 3] and [FIG. 4].

The electrodes 4 are configured to be in contact with the person P, and notably with the skin of the person P to acquire at least one measurement signal representative of a physiological electrical signal of the person P.

The physiological electrical signal is advantageously an electroencephalogram (EEG) of the person P.

The electrodes 4 can for example be produced from conductive polymers or metal.

The electrodes 4 are advantageously water-resistant.

“Water-resistance” is understood to mean that the quality of the electrodes is not likely to be degraded if they are placed in prolonged contact with the skin. In particular, electrodes 4 are still operational even after having undergone several washes, in a machine for example.

The electrodes 4 can comprise a structure in the form of micro-suckers allowing a suction-based hold between the electrode and the skin of the person P.

The electrodes 4 can also comprise a structure in the form of filaments, of the order of a micrometer or of ten or so micrometers, densely distributed on the surface of the electrode 4. These filaments make it possible to improve the contact between the electrode and the skin of the person P by Van der Waals interaction (inspired by the gecko).

Such structures make it possible to ensure a satisfactory contact between the electrode and the skin of the person P to acquire the measurement signal, or to ensure that the electrode is held in place while being disposed around the measurement signal acquisition zone.

The electrodes 4 can be fixed elements, for example glued or stitched with the three-dimensional fabric parts of the support element 2.

As a variant, the electrodes 4 form an integral part of the support element 2, notably of the inner face of the support element 2.

The expression “to form an integral part” is understood to mean in particular that the electrodes 4 are formed by the three-dimensional fabric. The electrodes are notably fabric electrodes, woven, knitted, and/or braided with the support element 2.

Such electrodes 4 are, for example, described in the document EP 1 858 581.

The electrodes 4 can be located in the support element 2, as illustrated in [FIG. 3] and [FIG. 4].

As a variant, the electrodes 4 can advantageously be more extensive on the surface, for example so as to be present over a significant part, even all, of a branch of the support element 2.

The support element 2 can, moreover, comprise at least one or even a plurality of acoustic transducers 5.

The acoustic transducers 5 are configured to emit an acoustic signal stimulating at least one inner ear of the person P.

The acoustic signal can be emitted by bone conduction.

As a variant, the acoustic signal can be emitted by means of a loudspeaker disposed at the level of an ear of the person P.

According to the invention of [FIG. 1], [FIG. 2], [FIG. 3], and [FIG. 4], two acoustic transducers 5 are disposed respectively in three-dimensional fabric parts 3c each configured to cover an ear of the person P once the support element 2 is worn.

The support element 2 can also comprise other types of sensors, such as a pulse oximeter, an accelerometer, a temperature sensor, a microphone (none of which are illustrated) or even any other type of sensor not described.

The support element 2 can notably comprise an elasticity sensor 6, illustrated in [FIG. 2], configured to measure the strain being exerted on, or the elastic elongation of, a three-dimensional fabric part 3b.

The elasticity sensor 6 thus makes it possible to know if the support element 2 is tightened too much or too little on the head of the person P.

Unit

The device 1 also comprises an embedded electronics conditioning and monitoring unit 7 (hereinafter called “unit”). The unit 7 is a rigid element.

The unit 7 is configured to be assembled with or disassembled from the support element 2. The unit 7, once assembled with the support element 2, makes it possible to obtain the device 1 in a state of operation.

The unit 7 can be assembled, advantageously reversibly, with the support element 2.

“Reversibly” is understood to mean that the unit 7 can be separated, notably electrically, from the support element 2, then assembled once again with the support element 2, repeatedly.

To this end, the support element 2 comprises a connection element 8, visible in [FIG. 4], configured to attach the unit 7 to the support element 2. The connection element 8 can be attached to the unit 7, notably electrically, by elastic fitting, by magnetic connection or by any other means.

Furthermore, the support element 2 also comprises an opening 9, visible also in [FIG. 4], configured to allow the unit 7 to be inserted into the support element 2.

According to the embodiment of [FIG. 4], one of the branches of the support element 2, for example the second branch 2b, can form a pocket or a housing 10 (in dotted lines in [FIG. 4]) in which the unit 7 can be placed.

The unit 7, once assembled with the support element 2, is configured to receive information from the electrodes 4, and can also be configured to control and monitor the emission by the acoustic transducers 5 of an acoustic signal.

The document WO 2016/083598 presents, for example, the general operation of the device 1 for stimulating the slow brainwaves.

The device 1 can, more generally, be used to analyze and/or monitor the brainwaves of the person P. The device 1 notably makes it possible to perform neurofeedback (also called neurotherapy) in order to improve the quality of sleep or help the person P go to sleep.

The unit 7 is also configured to communicate with a server or external device (not illustrated), advantageously wirelessly, for example by implementing a protocol such as Bluetooth or Wi-Fi.

The unit 7 can thus transfer the measurement signals to the external server or external device.

The unit 7 can also receive from the server or external device sounds to be emitted by the acoustic transducers 5.

### Electrical Conduction

In order for the device 1 to be in a state of operation once the unit 7 is assembled with the support element 2, it is necessary to electrically link the unit 7 to the other electrical elements of the support element 2, such as the electrodes 4, if necessary the acoustic transducers 5, or any other sensor, by means of electrical conduction elements.

To this end, according to a first embodiment, at least one three-dimensional fabric part forms a tubular channel or section 11, illustrated more particularly in [FIG. 2].

The channel 11 forms an integral part of the three-dimensional fabric part. The channel 11 can thus be deformed with the support element 2.

The channel 11 is configured to receive an electrical conduction element 12.

The electrical conduction element 12 thus makes it possible link an electrode 4 and/or an acoustic transducer 5 and/or also any other element to the unit 7.

The electrical conduction element 12 is advantageously a shielded cable.

A shielded cable is an electrical cable comprising one or more conductive elements 14 surrounded by one or more conductive layers. A conductive layer can be composed of a conductive braid 15, for example of braided strands of copper or of aluminum, and/or of a wound layer of aluminum or of conductive polymer 16. The conductive layer or layers act as a Faraday cage that makes it possible to reduce the electromagnetic disturbances originating from other electrical devices.

The use of a shielded cable is particularly advantageous for electrically linking the unit 7 to the electrodes 4. In fact, the electrodes 4 are capable of measuring one or more weak measurement signals, of the order of a microvolt. It is therefore necessary for the electromagnetic disturbances to be as limited as possible and not cause any degradation of these measurement signals.

As illustrated in [FIG. 7], a shielded cable can, for example, be a coaxial cable. A coaxial cable comprises a conductive central core 14, which can be single-stranded or multi-stranded, for example made of copper. The conductive central core 14 is surrounded by an insulating dielectric material 17. The dielectric material 17 is, itself, surrounded by a conductive sheet and/or braid 15, 16, then by an insulating and protective sheath 18.

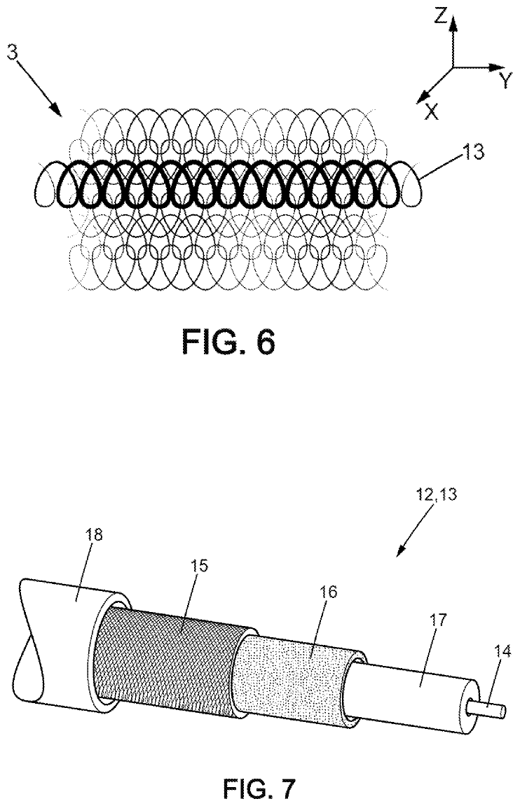

According to a second embodiment, at least one fabric part of the support element 2 comprises at least one electrical conduction element 13, illustrated more particularly in [FIG. 6]. The electrical conduction element 13 thus forms an integral part of the three-dimensional fabric part.

The expression “to form an integral part” is understood to mean in particular that the electrical conduction element 13 forms and/or is formed by the three-dimensional fabric part. The electrical conduction element 13 is notably woven, knitted, and/or braided with the three-dimensional fabric part.

Thus, the electrical conduction element 13 cannot be separated from the three-dimensional fabric part. The electrical conduction element 13 is notably inextricably linked or intermingled with the other fibers forming the three-dimensional fabric, such that the electrical conduction element 13 cannot be separated from the three-dimensional fabric part without causing it to be damaged, even destroyed.

According to one embodiment, the electrical conduction element 13 can be produced by means of conductive materials, such as a metal material, an intrinsically conductive polymer or a composite material.

Advantageously, the electrical conduction element 13 is a shielded cable, notably coaxial, as described above. The shielded cable then has dimensions, in particular a diameter, close to or equal to the diameter of the other fibers of the three-dimensional fabric part.

According to another embodiment, the electrical conduction element 13 can be produced by means of one or more fabric fibers coated with or impregnated by a conductive substance (such as silver, for example).

According to this second embodiment, it is not then necessary to use an electrical cable that is distinct from the support element 2, contrary to the first embodiment. A particularly simple and robust support element 2 is thus obtained.

More generally, according to the first and second embodiments described above, it is possible to electrically link the unit 7 to the elements of the support element 2 while conserving a support element 2 that is flexible, comfortable and easy to use.

### Advantage

As already stated above, the current devices for analyzing and monitoring brain activity do not give full satisfaction because they comprise rigid elements which can prove uncomfortable.

The support element 2 according to the invention advantageously comprises no, or few, rigid elements. The support element 2 is notably mostly made of fabric, even exclusively of fabric, notably of three-dimensional fabric.

“Mostly” is understood to mean that the support element 2 can however comprise certain rigid elements that do not contribute to the structure of the device 1, such as an electrical cable 12 or an electrical transducer 5.

The use of three-dimensional fabrics makes it possible to obtain a device 1 that is particularly comfortable for the person P, notably during a period of sleep.

Furthermore, the support element 2 can advantageously be water-resistant.

It is thus possible to disassemble the support element 2 and the unit 7 once the use of the device 1 has ended. The support element 2 can then be washed separately, notably in a washing machine. Once washed, the unit 7 can be assembled with the support element 2 in order for the device 1 to be once again in a state of operation.

The support element 2, being made of three-dimensional fabric, can have a certain specific rigidity. The support element 2 can notably be kept in shape without including any rigid reinforcement.

“Kept in shape” is understood to mean notably that the support element 2 keeps its shape when it is subjected to its own weight by being placed on a flat support.

As a variant, the support element 2 can include an additional reinforcement configured to give it a particular shape or keep it in shape when it is not worn by the person P.

1-10. (canceled)

11. A device for analyzing and/or monitoring brainwaves configured to be worn by a person, notably during a period when the person is sleeping, the device comprising a support element configured to at least partially surround a head of the person so as to be held thereon, wherein the support element comprises at least one three-dimensional fabric.

12. The device of claim 11, wherein the support element comprises a plurality of three-dimensional fabric parts.

13. The device of claim 11, wherein the support element is exclusively made of three-dimensional fabric.

14. The device of claim 11, wherein the support element is configured to be assembled reversibly with an embedded electronics conditioning and monitoring unit.

15. The device of claim 14, wherein the support element comprises at least one electrode configured to be in contact with the person to acquire at least one measurement signal representative of a physiological electrical signal of the person, the unit being configured to receive information from the electrode.

16. The device of claim 14, wherein the support element comprises at least one acoustic transducer configured to emit an acoustic signal stimulating an inner ear of the person, the acoustic transducer being disposed in a three-dimensional fabric part adapted to cover the ear of the person once the support element is worn by the person, the unit being configured to control and monitor the emission by the acoustic transducer of the acoustic signal.

17. The device of claim 15, wherein the unit is linked to the electrode by means of a shielded cable.

18. The device of claim 17, wherein the shielded cable forms an integral part of the three-dimensional fabric.

19. The device of claim 17, wherein the three-dimensional fabric forms at least one channel, the channel being configured to receive the shielded cable.

20. A kit for forming a device for analyzing and/or monitoring brainwaves as claimed in any one of the preceding claims, comprising a support element and an embedded electronics conditioning and monitoring unit, the unit being configured to be assembled reversibly with the support element.
